(19) United States
(12) Patent Application Publication
(10) Pub. No.: US 2022/0215552 A1
(43) Pub. Date: Jul. 7, 2022

(54) SYSTEM AND COMPUTER-IMPLEMENTED METHOD FOR MEDICAL IMAGE PROCESSING

(71) Applicants: Sachin Rohani, Maharashtra (IN); Dr. D.Y. Patil Vidyapeeth, Pune-Deemed to be Univ, Maharashta (IN)

(72) Inventors: Sachin Rohani, Maharashtra (IN); Urmila Abhijeet Patil, Maharashtra (IN)

(21) Appl. No.: 17/567,752

(22) Filed: Jan. 3, 2022

Related U.S. Application Data
(60) Provisional application No. 63/133,302, filed on Jan. 2, 2021.

(51) Int. Cl. G06T 7/00; G06V 40/10; G06T 5/00; G06F 40/205; G06V 10/74; G16H 10/60; G16H 10/20

(52) U.S. Cl. CPC G06T 7/0014; G06V 40/10; G06T 5/002; G06F 40/205; G06V 10/761; G10L 15/26; G16H 10/20; G06T 2207/30088; G06T 2200/24; G06T 2207/10024; G16H 10/60

(57) ABSTRACT

Disclosed herein is a system and method for medical image processing wherein an image of a body part of a subject patient is recorded and processed, by means of artificial intelligence based algorithms, to identify a type of dermatological condition. The subject patient is enabled to record one or more symptoms as experienced in relation to the said dermatological condition. The system compares the recorded image to a plurality of test images stored in a medical databank and identifies the best match while factoring in the associated symptoms as well. As a final output, the system displays an identified dermatological condition associated with the subject patient on a display device.

### CROSS REFERENCE TO RELATED APPLICATION

This application is a non-provisional application taking priority from the U.S. provisional patent application No. 63/133,302, titled “SYSTEM AND METHOD FOR MEDICAL IMAGE PROCESSING” and filed on 2 Jan. 2021.

### TECHNICAL FIELD

The present invention relates generally to technologies utilising image processing in medical science and more specifically, to a system and method for medical image processing using Artificial Intelligence (AI).

### BACKGROUND

In recent times, we have seen spurt in number of skin diseases spread across mankind on account of multiple bacterial and fungal infections. Many of these skin infections have been successfully cured subject to correct, speedy and efficient diagnosis by medical practitioners. However, many times, it becomes difficult for medical practitioners to accurately diagnose a dermatological condition. Worse, if they mis-diagnose and start medication on the misdiagnosed disease, that may lead to adverse reactions.

Conventionally, a dermatological infection or medical condition is diagnosed by a medical practitioner based on his experience and symptoms as detailed by patient in question. Often, a visual sight of infection may help the medical practitioner in identifying the underlying dermatological condition. Sometimes, the patient themselves ask for a specific treatment over tele-medicine channels and from pharmacy outlets.

However, there are several shortcomings of the abovementioned methods. The medical practitioner may fail to diagnose the dermatological condition if the same was not prior experienced by him. Further, mis-diagnosis can also occur as a result of human error and mis-judgment of visual sight of the infection since many infections have similar type of physical wound. Moreover, the symptoms detailed by the patient may not be accurate leading to said mis-diagnosis. Furthermore, the patient might start medications, bought over the counter, based on his own mis-judgment of dermatological condition, which might be dangerous to patient's health.

In light of the above-mentioned problems associated with existing methods and systems for identifying dermatological conditions in a patient, it is highly desirable to have a system and method for medical image processing that identifies an underlying medical condition based on image processing of a visual image associated with the dermatological condition and associated symptoms as recorded by a subject patient.

### SUMMARY

Embodiments of the present disclosure present technological improvements as solutions to one or more of the above-mentioned technical problems recognized by the inventor in conventional solutions.

According to a first aspect of the present invention there is provided a system for medical image processing. The system comprises a memory configured to store machine-readable instructions; and a processing unit operably connected with the memory unit. The processing unit obtains the machine-readable instructions from the memory unit, and is configured by the machine-readable instructions to receive an infection image file, associated with a patient, captured from an image recorder connected with a client device; receive a symptom file, associated with a patient, from the client device; process the infection image file to identify a body part and infection on the body part of the patient; process the symptom file to identify one or more symptoms in the patient based on a biomedical ontology; determine a best match record based on a comparison of the processed infection image and the identified one or more symptoms with each of a plurality of records in a medical databank; identify a dermatological condition of the patient, associated with the best match record; and display the dermatological condition on a graphical user interface associated with the client device.

In accordance with an embodiment of the present invention, the image recorder is a camera operable to capture colour images.

In accordance with an embodiment of the present invention, the infection image is pre-processed to remove noise and background.

In accordance with an embodiment of the present invention, the symptom file is generated using response, by the patient, to a questionnaire displayed on the graphical user interface.

In accordance with an embodiment of the present invention, the symptom file is generated using speech to text converter.

In accordance with an embodiment of the present invention, the medical databank comprises a plurality of records wherein each of the plurality of records relate to a specific dermatological condition.

In accordance with an embodiment of the present invention, the processing unit is further operable to display information associated with the identified dermatological condition on the graphical display unit.

In accordance with an embodiment of the present invention, the best match record is determined based on similarity score of infection image with images associated with each of the records of the medical databank being greater than a predetermined threshold.

In accordance with an embodiment of the present invention, the best match record is determined based on matching of one or more identified symptoms.

In accordance with an embodiment of the present invention, the symptoms file is parsed by a natural language processing algorithm to identify medical terminologies based on the biomedical ontology.

According to a second aspect of the present invention, there is provided a computer-implemented method for medical image processing. The computer-implemented method comprises receiving, at a processing unit, an infection Image file associated with a patient, captured from an image recorder connected with a client device; receiving, at a processing unit, a symptom file associated with the patient, from the client device; processing the infection image file to identify a body part and infection on the body part of the patient; processing the symptom file to identify one or more symptoms in the patient, based on a biomedical ontology; determining a best match record based on a comparison of the processed infection image and the identified one or more symptoms with each of a plurality of records in a medical databank; identifying a dermatological condition of the patient, associated with the best match record; and displaying the dermatological condition on a graphical user interface associated with the client device.

In accordance with an embodiment of the present invention, the image recorder is a camera operable to capture colour images.

In accordance with an embodiment of the present invention, the step of processing the infection image includes a step of pre-processing the infection image to remove noise and background.

In accordance with an embodiment of the present invention, the symptom file is generated using response, by the patient, to a questionnaire displayed on the graphical user interface.

In accordance with an embodiment of the present invention, the symptom file is generated using speech to text converter.

In accordance with an embodiment of the present invention, the medical databank comprises a plurality of records wherein each of the plurality of records relate to a specific dermatological condition.

In accordance with an embodiment of the present invention, the computer-implemented method further comprises a step of displaying information associated with the identified dermatological condition of the patient on the processing unit.

In accordance with an embodiment of the present invention, the step of determining a best match record includes generating a similarity score of infection image with images associated with each of the records of the medical databank and determining whether the similarity score is greater than a predetermined threshold.

In accordance with an embodiment of the present invention, the best match record is determined based on matching of one or more identified symptoms.

In accordance with an embodiment of the present invention, the computer-implemented further comprises a step of parsing the symptoms file by a natural language processing algorithm to identify medical terminologies based on the biomedical ontology.

Additional aspects, advantages, features and objects of the present disclosure would be made apparent from the drawings and the detailed description of the illustrative embodiments construed in conjunction with the complete specification that will follow.

It will be appreciated that features of the present disclosure are susceptible to being combined in various combinations without departing from the scope of the present disclosure.

### BRIEF DESCRIPTION OF DRAWINGS

So that the manner in which the above recited features of the present invention can be understood in detail, a more particular description of the invention, briefly summarized above, may have been referred by embodiments, some of which are illustrated in the appended drawings. It is to be noted, however, that the appended drawings illustrate only typical embodiments of this invention and are therefore not to be considered limiting of its scope, for the invention may admit to other equally effective embodiments.

These and other features, benefits, and advantages of the present invention will become apparent by reference to the following text figure, with like reference numbers referring to like structures across the views, wherein:

FIG. 1 illustrates an exemplary system for medical image processing, in accordance with an embodiment of the present invention;

FIG. 2 illustrates an exemplary method for medical image processing using system of FIG. 1, in accordance with an embodiment of the present invention; and

FIG. 3 illustrate an exemplary implementation of system and method of FIGS. 1 and 2 respectively, in accordance with an embodiment of the present invention.

### DESCRIPTION OF EMBODIMENTS

The present invention is described hereinafter by various embodiments with reference to the accompanying drawing, wherein reference numerals used in the accompanying drawing correspond to the like elements throughout the description.

While the present invention is described herein by way of example using embodiments and illustrative drawings, those skilled in the art will recognize that the invention is not limited to the embodiments of drawing or drawings described and are not intended to represent the scale of the various components. Further, some components that may form a part of the invention may not be illustrated in certain figures, for ease of illustration, and such omissions do not limit the embodiments outlined in any way. It should be understood that the drawings and detailed description thereto are not intended to limit the invention to the particular form disclosed, but on the contrary, the invention is to cover all modifications, equivalents, and alternatives falling within the scope of the present invention as defined by the appended claim. As used throughout this description, the word “may” is used in a permissive sense (i.e., meaning having the potential to), rather than the mandatory sense, (i.e., meaning must). Further, the words “a” or “an” mean “at least one” and the word “plurality” means “one or more” unless otherwise mentioned. Furthermore, the terminology and phraseology used herein is solely used for descriptive purposes and should not be construed as limiting in scope. Language such as “including,” “comprising,” “having,” “containing,” or “involving,” and variations thereof, is intended to be broad and encompass the subject matter listed thereafter, equivalents, and additional subject matter not recited, and is not intended to exclude other additives, components, integers or steps. Likewise, the term “comprising” is considered synonymous with the terms “including” or “containing” for applicable legal purposes.

Further, the various embodiments described herein below include specific method steps in an exemplary order but a wide variety of other such method steps could be implemented within the scope of the invention, including additional steps, omission of some steps, or performing the method in a different order.

The present invention aims to overcomes the drawbacks of the prior art by providing a system and method for medical image processing, wherein an image of a body part of a subject patient is recorded and processed, by means of artificial intelligence based algorithms, to identify a type of dermatological condition. The subject patient is enabled to record one or more symptoms as experienced in relation to the said dermatological condition. The system compares the recorded image to a plurality of test images stored in a medical databank and identifies the best match while factoring in the associated symptoms as well. As a final output, the system displays an identified dermatological condition associated with the subject patient on a display device.

The present invention is now described in detail with reference to accompanying drawings.

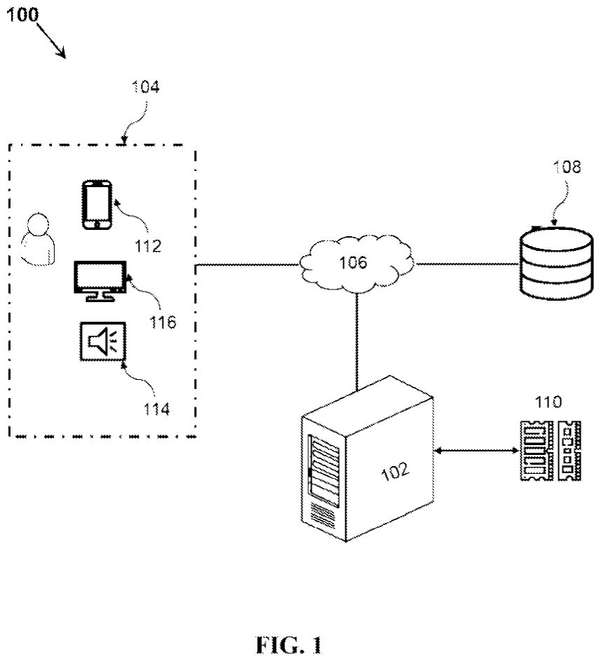

FIG. 1 depicts an exemplary system 100 for medical image processing, in accordance with an embodiment of the present invention. In an exemplary embodiment, the system 100 comprises a processing unit 102 communicably coupled, via one or more data communication networks 106, to a client device 104, a medical databank 108 and a memory 110.

In accordance with an embodiment of the present invention, the memory unit 110 is configured to store machine readable instructions. The machine-readable instructions may be loaded into the memory unit 110 from a non-transitory machine-readable medium, such as, but not limited to, CD-ROMs, DVD-ROMs and Flash Drives. Alternately, the machine-readable instructions may be loaded in a form of a computer software program into the memory unit 110. The memory unit in that manner may be selected from a group comprising, but not limited to, EPROM, EEPROM and Flash memory.

Furthermore, the processing unit 102 is operably connected with the memory unit 110. In various embodiments, the processing unit 102 is one of, but not limited to, microprocessor, a general-purpose processor, an application specific integrated circuit (ASIC) and a field-programmable gate array (FPGA). The processing unit 102 is capable of being used for local processing and/or cloud-based remote processing.

Moreover, the processing unit 102 may implement Artificial Intelligence (AI), Machine Learning (ML) and Deep Learning (DL) based technologies for, but not limited to, data analysis, collating data & presentation of data in real-time.

Further, the client device 104, as per the preferred embodiment, comprises at least an image recorder 112, a speech recorder 114, and a graphical display unit 116. The client device 104 can be in form of, but not limited to, a smartphone, mobile communication device, Tablet, Laptop or a desktop PC. The image recorder 112, as per the present disclosure, may be, but not limited to, a camera operable to capture images of a body part of a subject patient wherein the body part suffers from a dermatological condition. In case, the client device 104 is a smartphone, tablet, laptop or the like, then the image recorder 112 (i.e., the camera) is in-built in the client device 104. In case, the client device 104 is a PC or a laptop, then the image recorder 112 (such as webcam) may also be connected externally using a cable or wirelessly.

The image recorder 112 is configured to record the image of an infected body part in colour format, preferably RGB format. The image recorder 104 is further operable to store the recorded image as infection image in one or more readable format on the client device 104. As per the preferred embodiment of the present invention, the one or more readable format is one of .PNG, .JPEG, or .TIFF format. The infection image is transmitted, by the client device 104, to the processing unit 102 through the one or more data communication networks 106. The data communication network 106 can be a short-range communication network and/or a long-range communication network, wired or wireless communication network. The communication network may be implemented using a number of protocols, such as but not limited to, TCP/IP, 3GPP, 3GPP2, LTE, IEEE 802.x etc. The one or more data communication networks 106 may be wireless communication network selected from, but not limited to, Bluetooth, radio frequency, internet or satellite communication network providing maximum coverage. Preferably, the network is internet.

In accordance with an embodiment of the present invention, the medical data bank 108 may be a local storage (such as SSD, eMMC, Flash, SD card, etc) or a cloud-based storage. In any manner, the medical data bank 108 is envisaged to be capable of providing the data to the processing unit 102, when the data is queried appropriately using applicable security and other data transfer protocols.

The medical databank 108 comprises a plurality of records wherein each of the plurality of records relate to a specific dermatological condition. The plurality of record comprises one or more symptoms associated with the dermatological condition, an image of the said dermatological condition, an attribute master for the said image, a detailed description on the dermatological condition including the cause, risk factors, diagnosis, treatment and medications related to said dermatological condition. The attribute master records specific features of the image related an infection, such as reddishness, paleness, dissected etc. to name a few. A person skilled in the art shall appreciate that different dermatological conditions have different type of infection property and said properties are included in the attribute master file. These attributes help in identifying a type of infection/dermatological condition. In accordance with an embodiment of the present invention, the medical data bank may also include the plurality of records related to the specific dermatological condition, wherein a nomenclature of specific dermatological condition is according to Ayurveda and homeopathy. These may also be used for image processing in the present invention, as per the requirements of the respective users.

In another embodiment, the medical data bank 108 may also include prestored skin-based data in the form of videos and images of all skin-types of humans of multiple age groups, genders, countries and also multiple kinds of skin irregularities and disorders. In one embodiment, the Machine Learning and deep learning technology to prepare one or more trained machine learning models using the prestored data and the plurality of records in the data repository and enable real-time processing in the processing module.

In accordance with an embodiment of the present invention, the system 100 may be an embedded system (where all the components of the system 100 are provided as an integral unit) or a distributed system (wherein some of the components are provided separately, for example, processing unit 102 may be a cloud processing unit provided on a remote placed server and medical data bank 108 may also be a cloud-based storage).

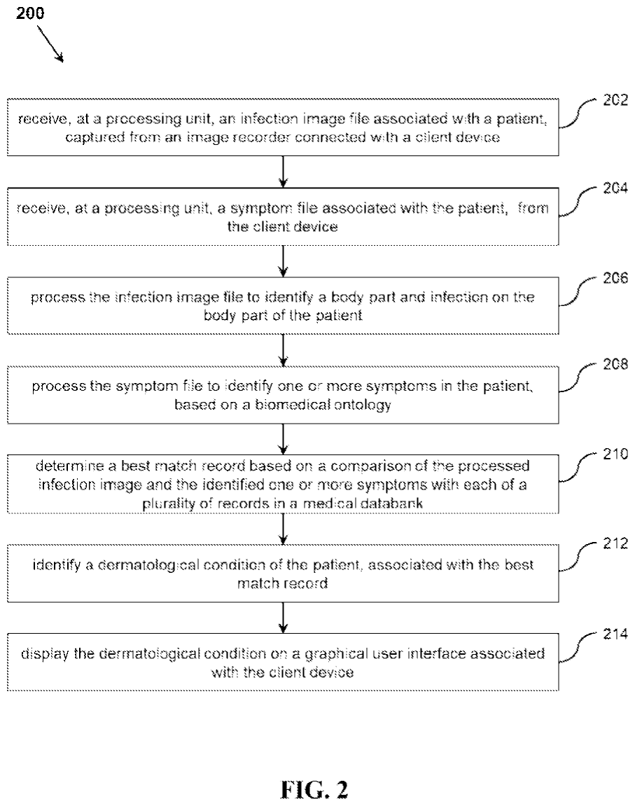

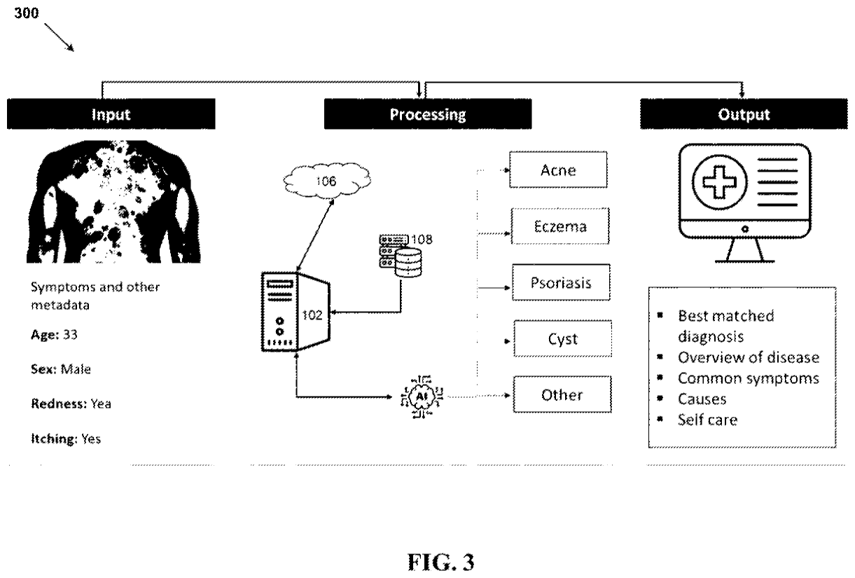

FIG. 2 illustrates a computer-implemented 200 for medical image processing, in accordance with an embodiment of the present invention. The working of the present invention would be more easily understood with reference to the exemplary implementation shown in FIG. 3. The computer-implemented begins at step 202 by receiving, at the processing unit 102, an infection image file associated with a patient, captured from the image recorder 112 connected with the client device 104. The image recorder 112, as per the present disclosure, may be, but not limited to, a camera operable to capture images of a body part of the patient wherein the body part suffers from a dermatological condition. The image recorder 112 is configured to record the image of an infected body part in colour format, preferably RGB format. The image recorder 104 is further operable to store the recorded image as infection image in one or more readable format on the client device 104. In a preferred embodiment of the present invention, the one or more readable format is one of, but not limited to, .PNG, .JPEG, or .TIFF format. The infection image is transmitted, by the client device 104, to the processing unit 102 through the one or more data communication networks 106. For example, as shown in the exemplary implementation in FIG. 3, an infection image of the patient along with a symptom file is received as input.

After that, at step 204, the processing unit 102 receives symptom file associated with the patient, from the client device 104. The client device 104 is configured to enable the patient to record one or more symptoms associated with the dermatological condition. In one embodiment, the speech recorder 114 on the client device 104 is used by the associated patient for the said purpose. The speech recorder 114 records the symptoms associated with the dermatological condition, as described by the patient orally. Optionally, the speech recorder 114 is configured to transform the recorded speech into text using a speech to text conversion algorithm. The text file is pre-processed and saved, in form of symptom file, on the client device 104 and transmitted to the processing unit 102 through the one or more data communication networks 106.

In an alternative embodiment of the present invention, the patient records the one or more symptoms associated with the dermatological condition by means of inputting text through the graphical display unit 116. Optionally, a questionnaire in a form of pre-defined questions may be displayed to the subject patient on the graphical display unit 116 and the response to the pre-defined questions may be recorded as the symptoms associated with the patient. The response, as recorded may be pre-processed, and saved in form of symptom file on the client device 104 and transmitted to the processing unit 102 through the one or more data communication networks 106.

Then, at step 206, the processing unit 102 processes the infection image file to identify a body part and infection on the body part of the patient. The processing unit 102 is configured to receive the infection image and the symptom file from the client device 104. The infection image relates to a body part infected with a dermatological condition and the symptom file relates to the symptoms related to the said dermatological condition. In an aspect of the present invention, the infection image is pre-processed to remove noise and background. Optionally, this is achieved through filtering and transformation of the infection image using one or more digital signal processing filters.

Once the infection image is pre-processed, the processing unit 102 is configured to employ an image detection algorithm to identify the body part from the pre-processed infection image. As an example, output of the image detection algorithm could be Leg, Hand or face or similar human body part. In an example embodiment, a subject patient suffering from fungal warts on palms may record an infection image of his palm. The processing unit 102 shall receive the infection image and pre-process the same. The image detection algorithm implemented on the pre-processed infection image will give the output as palm of hand.

In yet another aspect of the present disclosure, the image detection algorithm is configured to identify an infected portion on the identified body part. In the example embodiment, an area on the palm infected by the fungal warts may be highlighted or earmarked specifically by the image detection algorithm in its output file. Alternatively, the highlighted portion/infected portion may be saved as a cut-out infection image associated with the infection image.

Moreover, at step 208, the processing unit processes the symptom file received from the client device 104, to identify one or more symptoms in the patient based on biomedical ontology. The processing unit 102 employs one or more natural language processing algorithms on the symptoms file to extract specific medical terminologies from the free text by the patient. For example, if the patient describes spinning sensation as one of the symptoms, it may be processed and interpreted as dizziness by the natural language processing algorithm. The biomedical ontology comprising symptoms, their synonyms and grammatical variations is employed by the processing unit in conjunction with the natural language processing algorithms to determine exact medical terminologies for symptoms described by the subject patient. In operation, the symptom file is parsed and symptoms are identified based on the biomedical ontology and natural language processing.

Furthermore, at step 210, the processing unit 102 is configured to determine a best match record based on a comparison of the processed infection image and the identified one or more symptoms, with each of a plurality of records in a medical databank 108. The processing unit 102 is configured to compare the infection image with each of the plurality of records in the medical databank 108, generate a similarity score and identify at least one matching record with similar image. In one embodiment, the image processing algorithm used for said comparison is configured with a predefined threshold output. This results in only those records being displayed where the similarity score is greater than the set threshold output. Similarly, the identified symptoms are also compared with the symptoms of each of the matching record. Based on the said comparison, the processing unit 102 is operable to determine the best match record for the infection image.

Accordingly, at step 212, based on the above comparison, a dermatological condition of the patient associated with the best match record is retrieved from the medical databank the processing unit. The same can be referred from the exemplary implementation shown in FIG. 3, where the processing unit determined that the patient's skin has acne, eczema, psoriasis and cyst. In accordance with an embodiment of the present invention, if the patient desires, the name of dermatological/skin conditions may be suggested according to Ayurveda and homeopathy. This may be done using the plurality of records in the medical data bank, wherein the nomenclature of specific dermatological conditions are according to Ayurveda and homeopathy.

Then, at step 214, displayed to the subject patient on the graphical display unit 116 of the client device 104, as the diagnosed dermatological condition. Optionally, information about the diagnosed dermatological condition, may also be displayed on the processing unit 102 (in case, where the processing unit may be connected with a display unit) for detailed analysis and research.

In accordance with an embodiment of the present invention, along with the diagnosed dermatological condition, the processing unit 102 also provides overview of the disease, other related common symptoms, and common causes of the disease that may assist the patient and help him/her understand the condition in better manner.

In accordance with an embodiment of the present invention, the processing unit 102 is further configured to provide self-care tips, lifestyle changes, and possible modes of improving the diagnosed dermatological conditions, based prestored data in the medical data bank, as well as utilising AI and ML. For example: if a fungal infection is diagnosed by the present invention, then different probable modes of improvement may be suggested as per natural remedies, allopathy, ayurveda, and homeopathy. Kindly note that the present invention is only configured to provide above mentioned suggestions and no method of treatment or related medicines are prescribed by the present invention. Also, the patient may also be suggested to consult a doctor or a dermatologist if the diagnosed dermatological conditions are serious or of predetermined nature (that may be preconfigured to be categorised as ‘serious’, in the present invention).

In accordance with another embodiment of the present invention, the present invention may be provided as a mobile application, website or a software application to be installed/run on a smartphone, Tablet, Laptop or a desktop PC and used as a self-assessment tool by a respective user/patient. In such cases, the processing unit is further configured to store the records of skin diagnosis performed by the user in the medical data bank. So, whenever a new skin diagnosis is performed by the respective user, the system enables the user to monitor his/her progress in terms of the dermatological conditions, by comparing the previous images with the present images, that may be after treatment. For example: the system use criteria like skin color, thickness of skin, dryness of skin, scaling of skin etc. as a basis for the comparison.

It will be appreciated by a person skilled in the art that method steps 202-214 of the invention may also be performed without following any strict order, and by one or more computer processors executing a program tangibly embodied on a computer-readable medium to perform functions of the invention by operating on input and generating output. Suitable processors include, by way of example, both general and special purpose microprocessors. Generally, the processor receives (reads) instructions and data from a memory (such as a read-only memory and/or a random-access memory) and writes (stores) instructions and data to the memory. Storage devices suitable for tangibly embodying computer program instructions and data include, for example, all forms of non-volatile memory, such as semiconductor memory devices, including EPROM, EEPROM, and flash memory devices; magnetic disks such as internal hard disks and removable disks; magneto-optical disks; and CD-ROMs. Any of the foregoing may be supplemented by, or incorporated in, specially-designed ASICs (application-specific integrated circuits) or FPGAs (Field-Programmable Gate Arrays).

A computer can generally also receive (read) programs and data from, and write (store) programs and data to, a non-transitory computer-readable storage medium such as an internal disk (not shown) or a removable disk.

One or more components of the invention may be described as modules for the understanding of the specification. For example, a module may include self-contained component in a hardware circuit comprising of logical gate, semiconductor device, integrated circuits or any other discrete component. The module may also be a part of any software programme executed by any hardware entity for example processor. The implementation of module as a software programme may include a set of logical instructions to be executed by a processor or any other hardware entity.

Additional or less modules can be included without deviating from the novel art of this disclosure. In addition, each unit can include any number and combination of sub-units, and systems, implemented with any combination of hardware and/or software units.

Various modifications to these embodiments are apparent to those skilled in the art from the description and the accompanying drawings. The principles associated with the various embodiments described herein may be applied to other embodiments. Therefore, the description is not intended to be limited to the embodiments shown along with the accompanying drawings but is to be providing broadest scope of consistent with the principles and the novel and inventive features disclosed or suggested herein. Accordingly, the invention is anticipated to hold on to all other such alternatives, modifications, and variations that fall within the scope of the present invention and the appended claims.

1. A system for medical image processing, the system comprising:
a memory configured to store machine-readable instructions; and
a processing unit operably connected with the memory unit, the processing unit obtaining the machine-readable instructions from the memory unit, and being configured by the machine-readable instructions to:
receive an infection image file, associated with a patient, captured from an image recorder connected with a client device;
receive a symptom file, associated with a patient, from the client device;
process the infection image file to identify a body part and infection on the body part of the patient;
process the symptom file to identify one or more symptoms in the patient based on a biomedical ontology;
determine a best match record based on a comparison of the processed infection image and the identified one or more symptoms with each of a plurality of records in a medical databank;
identify a dermatological condition of the patient, associated with the best match record; and
display the dermatological condition on a graphical user interface associated with the client device.

2. The system of claim 1, wherein the image recorder is a camera operable to capture colour images.

3. The system of claim 1, wherein the infection image is pre-processed to remove noise and background.

4. The system of claim 1, wherein the symptom file is generated using response, by the patient, to a questionnaire displayed on the graphical user interface.

5. The system of claim 1, wherein the symptom file is generated using speech to text converter.

6. The system of claim 1 wherein the medical databank comprises a plurality of records wherein each of the plurality of records relate to a specific dermatological condition.

7. The system of claim 1 wherein the processing unit is further operable to display information associated with the identified dermatological condition on the graphical display unit.

8. The system of claim 1, wherein the best match record is determined based on similarity score of infection image with images associated with each of the records of the medical databank being greater than a predetermined threshold.

9. The system of claim 8, wherein the best match record is determined based on matching of one or more identified symptoms.

10. The system of claim 1, wherein the symptoms file is parsed by a natural language processing algorithm to identify medical terminologies based on the biomedical ontology.

11. A computer-implemented method for medical image processing, the computer-implemented method comprising:
receiving, at a processing unit, an infection image file associated with a patient, captured from an image recorder connected with a client device;
receiving, at a processing unit, a symptom file associated with the patient, from the client device;
processing the infection image file to identify a body part and infection on the body part of the patient;
processing the symptom file to identify one or more symptoms in the patient, based on a biomedical ontology;
determining a best match record based on a comparison of the processed infection image and the identified one or more symptoms with each of a plurality of records in a medical databank;
identifying a dermatological condition of the patient, associated with the best match record; and
displaying the dermatological condition on a graphical user interface associated with the client device.

12. The computer-implemented method of claim 11, wherein the image recorder is a camera operable to capture colour images.

13. The computer-implemented method of claim 11, wherein the step of processing the infection image includes a step of pre-processing the infection image to remove noise and background.

14. The computer-implemented method of claim 11, wherein the symptom file is generated using response, by the patient, to a questionnaire displayed on the graphical user interface.

15. The computer-implemented method of claim 11, wherein the symptom file is generated using speech to text converter.

16. The computer-implemented method of claim 11 wherein the medical databank comprises a plurality of records wherein each of the plurality of records relate to a specific dermatological condition.

17. The computer-implemented method of claim 11, further comprising a step of displaying information associated with the identified dermatological condition of the patient on the processing unit.

18. The computer-implemented method of claim 11, wherein the step of determining a best match record includes generating a similarity score of infection image with images associated with each of the records of the medical databank and determining whether the similarity score is greater than a predetermined threshold.

19. The computer-implemented method of claim 18, wherein the best match record is determined based on matching of one or more identified symptoms.

20. The computer-implemented method of claim 11, further comprising a step of parsing the symptoms file by a natural language processing algorithm to identify medical terminologies based on the biomedical ontology.
